(19) United States
(12) Patent Application Publication
(10) Pub. No.: US 2018/0294051 A1
(43) Pub. Date: Oct. 11, 2018

(54) CLINICAL SUPPORT SYSTEM AND METHOD

(71) Applicant: The University of British Columbia, Vancouver (CA)

(72) Inventors: Martin DAWES, Vancouver (CA); Diana DAWES, Vancouver (CA)

(21) Appl. No.: 15/570,662

(22) PCT Filed: Apr. 29, 2016
(86) PCT No.: PCT/CA2016/050495

§ 371 Date: Oct. 30, 2017

Related U.S. Application Data
(60) Provisional application No. 62/154,540, filed on Apr. 29, 2015.

(51) Int. Cl. G16H 20/30; G16H 10/60; G16H 50/30; G16H 50/20

(52) U.S. Cl. CPC G16H 20/30; G16H 50/20; G16H 50/30; G16H 10/60

(57) ABSTRACT

There is provided a computer-implemented method and device for providing clinical support. A user may input to the device a medical condition as well as a number of patient characteristics. The device may use a logic tree to determine, based on the medical condition, an initial list of therapeutic treatment options, such as specific drug(s) and associated dosage regimes, for treating the medical condition. The initial list may be refined by using a logic tree to determine, based on the patient characteristic(s), which of the therapeutic treatment options may not be appropriate for the patient, and such therapeutic treatment options may be removed and/or replaced with alternative therapeutic treatment options.

### TECHNICAL FIELD

The present disclosure is directed at methods and systems for providing clinical decision support. More particularly, the present disclosure is directed at methods and systems for providing patient centered clinical support using genetic information.

### BACKGROUND

Family physicians often deliver the majority of healthcare. For example in Canada family physicians deliver 85% of healthcare; however, they are increasingly unable to manage drug therapy for many patients. Each family physician writes an average of 20,000 prescriptions per year for over 233 different drugs. It is difficult to predict, when giving a patient a new prescription, whether that individual will receive the desired benefit (effective dose) from the medication, whether they will suffer harmful side effects from the medication, or both. Non-invasive genetic tests can help identify those more likely to benefit, and those more likely to be harmed by medications. However, this genetic information has generally not been incorporated into patient centered medication prescribing processes.

### SUMMARY

According to one aspect of the invention, there is provided a computer-implemented method for providing clinical support. The method comprises receiving at a device an indication of a medical condition. The method further comprises accessing with the device a database comprising a list of medical conditions, a list of therapeutic treatment options for treating each of the medical conditions, a list of patient characteristics, and a list of modifying factors of each patient characteristic on each therapeutic treatment option. The method further includes generating at the device an initial therapeutic treatment options list based on the indication of the medical condition, the list of medical conditions and the list of therapeutic treatment options. The method further includes receiving at the device at least one patient characteristic. The method further includes generating at the device a list of patient specific modifying factors based on the at least one patient characteristic and the list of modifying factors of each patient characteristic on each therapeutic treatment option. The method further includes generating at the device a final therapeutic treatment options list based on the initial therapeutic treatment options list and the list of patient specific modifying factors.

The indication of a medical condition may be received at the device via user input. The indication of a medical condition may include an explicit indication of the medical condition, or else may include information allowing deduction (for example either by a physician or by the device itself) of a medical condition. For example the indication may include a name of a drug or some other medicament, and based on this information a medical condition may be deduced. More than one indication of a medical condition may be received at the device, such that more than one medical condition may be deduced.

As used herein, a list may comprise one or more elements (and in some cases may comprise nil elements). Each medical condition may be treatable by one or more therapeutic treatment options, and a particular therapeutic treatment option may be used to treat more than one medical condition.

Each patient characteristic in the list of patient characteristics may comprise information on at least one of: a diagnosis, an age, a gender, a functional severity of a disease, a pharmacological status with current drugs, a medication history, biophysical information, an additional medical condition, and an allergy.

A patient characteristic may be any characteristic or piece of information which may have an impact on the benefit or harm of a therapeutic treatment option. For example, a patient characteristic may comprise information relating to an independent medical condition from which the patient is currently suffering. This independent medical condition may have an impact on which therapeutic treatment options may be suitable for treating the medical condition under investigation. For example, certain therapeutic treatment options may be inappropriate for treating the medical condition under investigation in view of the other, independent medical condition from which the patient is suffering. Thus, different patient specific modifying factors may be generated based on this independent medical condition.

Each patient characteristic in the list of patient characteristics may comprise genetic information. The genetic information may comprise a genetic variant. The genetic variant may render the patient predisposed towards greater harm or benefit from a particular therapeutic treatment option.

Generating the initial therapeutic treatment options list may comprise identifying in the list of medical conditions a particular medical condition based on the received indication of a medical condition. Generating the initial therapeutic treatment options list may further comprise using one or more logic trees to identify in the list of therapeutic treatment options one or more particular therapeutic treatment options, the one or more particular therapeutic treatment options being suitable for treating the particular medical condition.

Generating the list of patient specific modifying factors may comprise using one or more logic trees to identify in the list of modifying factors one or more particular modifying factors, the one or more particular modifying factors being associated with at least one of the received one or more patient characteristics and a therapeutic treatment option in the initial therapeutic treatment options list.

Thus, for a given therapeutic treatment option in the initial therapeutic treatment options list, a number of patient specific modifying factors may be generated (these patient specific modifying factors being selected from the list of modifying factors) based on whether any of the patient characteristics received at the device have an impact on the benefit or harm of the given therapeutic treatment option.

The list of patient specific modifying factors may be generated based not only on the list of patient characteristics and the list of modifying factors, but also on the basis of the initial therapeutic treatment options list. That is to say, a particular therapeutic treatment option in the initial therapeutic treatment options list may affect which modifying factors are selected for the list of patient specific modifying factors.

A logic tree may identify associations between one or more of: a medical condition in the list of medical conditions and a therapeutic treatment option in the list of therapeutic treatment options; a modifying factor in the list of modifying factors, a patient characteristic in the list of patient characteristics and a therapeutic treatment option in the list of therapeutic treatment options; and a modifying factor in the list of modifying factors, a patient characteristic in the list of patient characteristics, a therapeutic treatment option in the list of therapeutic treatment options and a medical condition in the list of medical conditions.

The one or more logic trees may be stored in the database.

A therapeutic treatment option may comprise a drug and a dosage regime of the drug.

A modifying factor may comprise an adjustment of a therapeutic treatment option.

The adjustment may comprise one of the following: a removal of the therapeutic treatment option from the initial therapeutic treatment options list; a modification of a dosage regime comprised in the therapeutic treatment option; and a replacement of the therapeutic treatment option in the initial therapeutic treatment options list with another therapeutic treatment option from the list of therapeutic treatment options.

Thus, there is provided a computer-implemented method and device for providing clinical support. A user may input to the device a medical condition as well as a number of patient characteristics. The device may use a logic tree to determine, based on the medical condition, an initial list of therapeutic treatment options, such as specific drug(s) and associated dosage regimes, for treating the medical condition. The initial list may be refined by using a logic tree to determine, based on the patient characteristic(s), which of the therapeutic treatment options may not be appropriate for the patient, and such therapeutic treatment options may be removed and/or replaced with alternative therapeutic treatment options.

A modifying factor may be based on evidence of harm and/or benefit linking the patient characteristic to the therapeutic treatment option.

The modifying factor may be further based on evidence of harm and/or benefit linking the patient characteristic to a medical condition suitable for being treated by the therapeutic treatment option.

Receiving the at least one patient characteristic may comprise accessing an electronic health record of a patient. For example, a patient's electronic health record may have stored thereon a list of patient characteristics for the particular patient. The device may access the electronic health record in order to obtain the list of patient characteristics. Alternatively, the at least one patient characteristic may be received at the device via user input.

Generating the final therapeutic treatment options list may comprise applying the list of patient specific modifying factors to the initial therapeutic treatment options list. Thus, the initial therapeutic treatment options list may be modified to account for the patient specific modifying factors. For example, those therapeutic treatment options in the initial therapeutic treatment options list deemed, on the basis of the patient specific modifying factors, to cause harm to the patient may be removed, amended or else replaced with other, safer therapeutic treatment options.

The method may further comprise using the device to cause display of the final therapeutic treatment options list on a display.

In a further aspect of the disclosure, there is provided a system for providing clinical support. The system comprises a database comprising a list of medical conditions, a list of therapeutic treatment options for treating each of the medical conditions, a list of patient characteristics, and a list of modifying factors of each patient characteristic on each therapeutic treatment option. The system further comprises a device comprising memory and a processor. The processor is configured to receive an indication of a medical condition and at least one patient characteristic. The processor is further configured to access the database. The processor is further configured to generate an initial therapeutic treatment options list based on the indication of the medical condition, the list of medical conditions and the list of therapeutic treatment options. The processor is further configured to generate a list of patient specific modifying factors based on the at least one patient characteristic and the list of modifying factors of each patient characteristic on each therapeutic treatment option. The processor is further configured to generate a final therapeutic treatment options list based on the initial therapeutic treatment options list and the list of patient specific modifying factors.

According to one aspect of the disclosure, there is provided a method for providing clinical support. The method includes receiving a diagnosis, generating an initial therapeutic options list, the initial therapeutic options list comprising at least one selected therapeutic treatment option for treating a medical condition indicated by the diagnosis from a database comprising logic trees, a list of medical conditions, therapeutic treatment options for treating each of the medical conditions, the evidence of benefit and harm associated with each therapeutic treatment option, and a list of modifying factors. The method also includes obtaining at least one patient characteristic, generating a list of at least one patient specific factor from the list of modifying factors in the database based on the effect of the patient characteristics on the selected therapeutic treatment options, generating a final therapeutic options list comprising at least one selected therapeutic treatment option for treating the medical condition, wherein the selected therapeutic treatment option is selected based on the patient specific factor, and sending the final therapeutic options list either directly to a health professional, directly to a patient or to an electronic health record.

The method may also include displaying the final therapeutic options list on a display. This may be visible to patients and healthcare professionals, to enable patient centred care.

The at least one patient characteristic may include information on at least one of an additional diagnosis, age, gender, functional severity of disease, pharmacological status with current drugs, medication history, biophysical information, additional medical conditions, genetic variants, and allergies. Patients, or healthcare professionals may hold and supply this information.

The modifying factors may be factors based on the effect of at least one of an additional diagnosis, age, gender, functional severity of disease, pharmacological status with current drugs, medication history, biophysical information, additional medical conditions, genetic variants, and allergies on the therapeutic treatment options. Patients or healthcare professionals may hold and supply this information.

The modifying factors may include factors based on the effect of one or more additional therapeutic treatment options on the therapeutic treatment options. Patients or healthcare professionals may hold and supply this information.

The method may include revising the patient characteristics.

According to another aspect of the disclosure, a method for providing clinical support is provided. The method includes receiving a diagnosis and generating an initial therapeutic options list, wherein the initial therapeutic options list includes at least one selected therapeutic treatment option for treating a medical condition indicated by the diagnosis and innate harms associated with each of the at least one selected therapeutic treatment option and wherein the initial therapeutic options list is generated from logic trees and a database that includes a list of medical conditions, therapeutic treatment options for treating each of the medical conditions, innate harms associated with each therapeutic treatment option, and patient specific harms based on the interaction of the therapeutic treatment options with patient characteristics. The method also includes retrieving at least one patient characteristic, generating a list of at least one patient specific harm based on the effect of the patient characteristics on the at least one selected therapeutic treatment option from the patient specific harms on the database, generating a final therapeutic options list comprising the at least one selected therapeutic treatment option, associated innate harms, and the at least one patient specific harm by combining the initial therapeutic options list with the list of at least one patient specific harm, and sending the final therapeutic options list either directly to a health professional, directly to a patient or to an electronic health record.

The method may also include the use of logic trees for removing from the final therapeutic options list selected therapeutic treatment options associated with an innate harm and a patient specific harm or patient specific harms from more than one patient characteristic if there are selected therapeutic treatment options available that are associated with a total of one or less innate harms and patient specific harms.

The method may also include modifying selected therapeutic treatment options on the first list that have an associated innate harm or patient specific harm by modifying dosages according to dosage modification guidelines in the database.

According to another aspect of the disclosure, a clinical support system is provided. The system includes a computer readable memory having stored thereon a database comprising a list of medical conditions, therapeutic treatment options for treating each of the medical conditions, logic trees that are based on the evidence of benefit and harm associated with each therapeutic treatment option, and a list of modifying factors, the modifying factors including factors based on the effect of genetic variants on the therapeutic treatment options. The system also includes a processor operably coupled to the memory and an application stored on the computer readable memory for execution by the processor for receiving a diagnosis input, generating an initial therapeutic options list, the initial therapeutic options list comprising at least one selected therapeutic treatment option for treating a medical condition indicated by the diagnosis and probabilities of benefit and harm for each selected therapeutic treatment option from the database, obtaining at least one patient characteristic, wherein the at least one patient characteristic includes information that guides therapeutic decision making, generating a list of at least one patient specific factor from the list of modifying factors in the database based on a logic tree that assesses the effect of the patient characteristics on the selected therapeutic treatment options, generating a final therapeutic options list comprising at least one selected therapeutic treatment option for treating the medical condition, wherein the associated evidence of benefit and harm are obtained by using the at least one patient specific factors to modify therapeutic options list before sending directly to a health professional, directly to a patient or to an electronic health record.

The system may also include a client application stored on a computer for execution by the computer for receiving the diagnosis input from a user and sending the diagnosis input to the application.

According to another aspect of the invention, a computer program product for providing clinical support is provided. The computer program product includes a non-transitory computer-readable medium having computer-readable code embodied therein executable by a processor for performing a method for providing clinical support. The method includes receiving a diagnosis, generating an initial therapeutic options list, the initial therapeutic options list comprising at least one selected therapeutic treatment option for treating a medical condition indicated by the diagnosis and evidence of benefit and harm for each selected therapeutic treatment option from a database of logic trees comprising a list of medical conditions, therapeutic treatment options for treating each of the medical conditions, the evidence of benefit and harm associated with each therapeutic treatment option, and a list of modifying factors. The method also includes obtaining at least one patient characteristic from an electronic health record, wherein the at least one patient characteristic includes genetic information. The method also includes generating a list of at least one patient specific factor from the list of modifying factors in the database based on the effect of the patient characteristics on the selected therapeutic treatment options, wherein the modifying factors include factors based on the effect of genetic variants on the therapeutic treatment options. The method also includes generating a final therapeutic options list comprising at least one selected therapeutic treatment option for treating the medical condition, wherein the selected therapeutic treatment option is selected based on the patient specific factor, and sending the final therapeutic options list directly to a health professional, directly to a patient or to an electronic health record.

According to another aspect of the disclosure, a genomic assay for genetic variants to provide genetic information for clinical support in a clinical support system is provided. The system includes a computer readable memory having stored thereon a database comprising a list of medical conditions, therapeutic treatment options for treating each of the medical conditions, logic trees that are based on the evidence of benefit and harm associated with each therapeutic treatment option, and a list of modifying factors, the modifying factors including the genetic information comprising factors based on the effect of the genetic variants on the therapeutic treatment options. The system also includes a processor operably coupled to the memory and an application stored on the computer readable memory for execution by the processor for receiving a diagnosis input, generating an initial therapeutic options list, the initial therapeutic options list comprising at least one selected therapeutic treatment option for treating a medical condition indicated by the diagnosis and evidence of benefit and harm for each selected therapeutic treatment option from the database, obtaining at least one patient characteristic from an electronic health record, wherein the at least one patient characteristic includes information that guides therapeutic decision making, generating a list of at least one patient specific factor from the list of modifying factors in the database based on a logic tree that assesses the effect of the patient characteristics on the selected therapeutic treatment options, generating a final therapeutic options list comprising at least one selected therapeutic treatment option for treating the medical condition, wherein the selected therapeutic treatment option is selected based on the patient specific factor, and sending the final therapeutic options list either directly to a health professional, directly to a patient or to an electronic health record.

According to another aspect of the disclosure, a use of a genomic assay for genetic variants to provide genetic information for clinical support in a clinical support system is provided. The system includes a computer readable memory having stored thereon a database comprising a list of medical conditions, therapeutic treatment options for treating each of the medical conditions, logic trees that are based on the evidence of benefit and harm associated with each therapeutic treatment option, and a list of modifying factors, the modifying factors including the genetic information comprising factors based on the effect of the genetic variants on the therapeutic treatment options. The system also includes a processor operably coupled to the memory. The system also includes an application stored on the computer readable memory for execution by the processor for receiving a diagnosis input, generating an initial therapeutic options list, the initial therapeutic options list comprising at least one selected therapeutic treatment option for treating a medical condition indicated by the diagnosis and evidence of benefit and harm for each selected therapeutic treatment option from the database, obtaining at least one patient characteristic from an electronic health record, wherein the at least one patient characteristic includes information that guides therapeutic decision making, generating a list of at least one patient specific factor from the list of modifying factors in the database based on a logic tree that assesses the effect of the patient characteristics on the selected therapeutic treatment options, generating a final therapeutic options list comprising at least one selected therapeutic treatment option for treating the medical condition, wherein the selected therapeutic treatment option is selected based on the patient specific factor, and sending the final therapeutic options list either directly to a health professional, directly to a patient or to an electronic health record.

### BRIEF DESCRIPTION OF THE DRAWINGS

In the accompanying drawings, which illustrate non-limiting embodiments of the invention,

FIG. 1 is a schematic of a clinical support system, according to a first embodiment;

FIG. 2 is a block diagram of a method for providing clinical support, according to another embodiment;

FIG. 3 is a screen shot of a graphical user interface showing a final therapeutic treatment options list embedded in a display of an electronic health record;

FIGS. 4A and 4B are examples of using a method for providing clinical support, according to another embodiment; and

FIG. 5 is an example of using a method for providing clinical support, according to another embodiment.

### DETAILED DESCRIPTION

The more medications a patient takes the more likely that patient is to experience an adverse drug event (“ADE”). Twenty percent of Canadians over the age of 65 take 10 or more drugs and are at significant risk for an ADE. To further complicate therapeutic decision-making processes, one individual may metabolize a drug up to 45 times more effectively than another individual due to a difference in their genetics (i.e., in one or more of the nucleotides within their DNA sequence). This variation in metabolism may result in a major toxic event and that may not result in any therapeutic effect or may even end in death. The present inventors have appreciated the challenges posed by the increasing numbers of patients taking multiple prescription drugs as well as the wide variation of individual, genetically determined responses to medications.

The present disclosure discloses a novel, user-driven decision support system that incorporates genetic information that may be used within a typical family practice consultation, or a pharmacist consultation, or by a patient prior to consultation. Family physicians, pharmacists, and patients may benefit from a genetically-informed medication management tool embodied either as a web-tool, a mobile application or integrated into their electronic health record (“EHR”) system to provide safer, more accurate therapeutic treatment advice for individual patients. An EHR is a record of medical and health data specific to a patient, including information on topics such as that patient's medical history, therapeutic history, and biophysical measurements. Unlike current EHR prescription modules, the subject matter disclosed herein may use information on a patient's genetic variations and known drug ADEs, as well as other relevant patient data, to guide more personalised prescribing. As used herein, the term electronic health record includes electronic records that may be referred to as electronic medical records (“EMR”), personal health records (“PHR”), and electronic health records.

The decision support system uses a method that starts with a diagnosis, drug, or other indication of a medical condition, and then, using a tree like structure such as a logic tree, produces a list of evidence-based therapeutic therapy or drug options that takes into account one or more patient characteristics such as age, gender, functional severity of a disease, pharmacological status with current drugs, allergies, and genetic status or information. The logic tree structure uses evidence of effectiveness and harm gathered from resources such as national disease management guidelines, systematic reviews, randomized controlled trials, and product monographs.

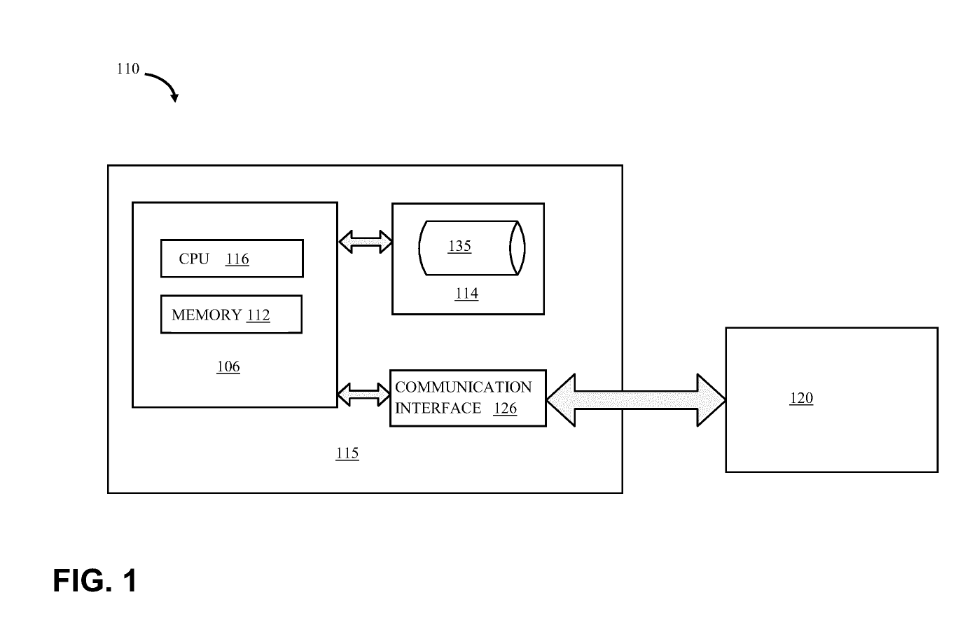

Referring to FIG. 1, a clinical support system 110 is shown. The system 110 includes an application server 115 and a client 120. The application server 115 comprises a computer 106 that may comprise one or more processors or microprocessors, such as a central processing unit (CPU) 116, which is depicted. The CPU 116 performs arithmetic calculations and control functions to execute software stored in an internal memory 112, such as one or both of random access memory (RAM) and read only memory (ROM), and possibly additional memory 114. The additional memory 114 may comprise, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 114 may be physically internal to the computer 106, or external as shown in FIG. 1, or both. The CPU 116 may retrieve items, such as applications and data lists, stored on the additional memory 114 and move them to the internal memory 112, such as RAM, so that they may be executed or to perform operations on them.

The application server 115 may also comprise other similar means for allowing computer programs or other instructions to be loaded. Such means can comprise, for example, a communications interface 126 that allows software and data to be transferred between the application server 115 and external systems and networks, such as the client 120. Examples of the communications interface 126 comprise a modem, a network interface such as an Ethernet card, a wireless communication interface, bar code reader, or a serial or parallel communications port. Software and data transferred via the communications interface 126 are in the form of signals which can be electronic, acoustic, electromagnetic, optical, or other signals capable of being received by the communications interface 126. Multiple interfaces, of course, can be provided on the application server 115.

In some embodiments (not depicted), the application server 115 may also comprise a display, a keyboard, pointing devices such as a mouse, and a graphical processing unit (GPU). The various components of the application server 115 are coupled to one another either directly or indirectly by shared coupling to one or more suitable buses.

The client 120 may be a personal computer (PC) in, for example, a doctor's office or a pharmacy, and may include all of the components described above for the application server 115. For example, the client 120 may include a computer, a display, external devices, and input devices such as a keyboard, pointing devices, a touchpad, or a touch screen. An I/O interface administers control of the display, keyboard, external devices and other components. The computer also comprises a GPU. The GPU may also be used for computational purposes as an adjunct to, or instead of, a CPU, for mathematical calculations. In some embodiments, the client 120 may be, for example, a laptop, a tablet computer, a handheld device such as a mobile telephone, or a computer terminal connected to a server.

The additional memory 114 includes a database 135. The database 135 stores a list of medical conditions and a list of therapeutic treatment options for treating each of the medical conditions. The database 135 also has stored thereon a list of patient characteristics, and a list of modifying factors of each patient characteristic on each therapeutic treatment option. In some embodiments, the database 135 may also comprise any of a drug-drug interaction database, a drug-disease interaction database, and disease management logic trees. The database 135 may also comprise comparative costs of medications a well as, for example, renal, liver, genetic and drug-drug dose adjustments. The database 135 may have an SQL database structure.

In certain embodiments, the modifying factors are based on the effect of patient characteristics on the therapeutic treatment options. For example, the modifying factors may include factors based on the effect of genetic variants on the therapeutic treatment options. In other embodiments, the modifying factors may include factors based on the effect on the therapeutic treatment options of at least one of age, gender, functional severity of a disease, pharmacological status with current drugs, medication history, biophysical information, such as renal and liver problems, co-morbidities or additional medical conditions, and allergies. In other embodiments, other suitable modifying factors may be included.

The database 135 may contain disease specific logic trees that incorporate data from the lists identified above. Such logic trees are derived from evidence such as, for example, national and international guidelines, systematic reviews, randomised controlled trials and other similar evidence to inform potential benefit and harm of the therapeutic treatment options.

Medication history may be included as a modifying factor because interactions of drugs with other drugs may be a factor affecting the efficacy of a therapeutic treatment option. The database 135 may include modifying factors based on the interaction of a therapeutic treatment option with a patient characteristic indicating a current medication being taken.

In some embodiments, the database 135 may include direct benefits and harms associated with each therapeutic treatment option, and patient specific benefits and harms based on the effect of a patient characteristic on a therapeutic treatment options. In other embodiments, the database 135 may include other information required for establishing the optimal medication including disease specific functional variables, such as the severity of the disease.

In some embodiments, the database 135 may comprise multiple databases. For example, the database 135 may comprise a first database comprising the list of medical conditions, the list of therapeutic treatment options for treating each of the medical conditions, and logic trees that incorporate the evidence of benefit and harm associated with each therapeutic treatment option, and a second database comprising the list of modifying factors.

In one embodiment, the therapeutic treatment options on the database 135 may include logic trees that incorporate evidence from a sub group of drugs identified by the Screening Tool of Older Person's Prescriptions and Screening Tool to Alert doctors to Right Treatment (STOPP START) process and the medical conditions that the drugs are used to treat (see, for example, Gallagher P, Ryan C, Byrne S, Kennedy J, & O'Mahony D 2008, ‘STOPP (Screening Tool of Older Person's Prescriptions) and START (Screening Tool to Alert doctors to Right Treatment). Consensus validation.’, International Journal of Clinical Pharmacology and Therapeutics, vol. 46, no. 2, pp. 72-83). The STOPP START criteria have been developed to support clinicians prescribe drugs more rationally to their elderly patients. These criteria consist of 78 recommendations that, when applied, support evidence-based, individualized prescribing practices to patients aged 65 and over. The criteria take into account a range of salient patient features to predict potentially inappropriate prescriptions. Their focus on the domain of higher risk patients in lower risk primary care environments is conducive to strategic healthcare innovation with respect to both improved health outcomes and lowered healthcare costs.

Alternatively, the therapeutic treatment options may include groups of therapeutic treatment options selected from drugs included in other guidelines and criteria designed to reduce inappropriate prescribing in the elderly and to aid prescribers use a rational approach to drug prescriptions.

The database 135 may include therapeutic treatment options selected from drugs known to have harmful side effects in addition to their known benefits.

As discussed above, the database 135 includes modifying factors. These factors are based on patient characteristics that may include any of age, gender, functional severity of a disease, pharmacological status with current drugs, medication history, biophysical information, co-morbidities, and allergies. Other patient characteristics may be used. For example, a modifying factor may be based on the presence of a Non-Steroidal Anti-Inflammatory (NSAID) drug (the patient characteristic) when considering the use of a Selective Serotonin Reuptake Inhibitor (SSRI) (the therapeutic treatment option) for the treatment of depression. The SSRI increases the risk of bleeding and has a number needed to harm (“NNH”) of 411 alone but in combination with a NSAID it is 106 (Loke, Y. K., Trivedi, A. N. & Singh, S., 2008. Meta-analysis: gastrointestinal bleeding due to interaction between selective serotonin uptake inhibitors and non-steroidal anti-inflammatory drugs. Alimentary pharmacology & therapeutics, 27 (1), pp. 31-40.). In another example, a modifying factor may be based on the presence of a penicillin allergy (the patient characteristic) when selecting an antibiotic (the therapeutic treatment option) for an acute infective exacerbation of Chronic Obstructive Pulmonary Disease. Modifying factors based on the effects of genetic variants on therapeutic treatment options are also included. Genetic variants (a patient characteristic) may influence the speed of metabolism of a drug and so may result in increased or decreased bioavailability. Increased or decreased bioavailability may be dealt with through dosage adjustments and/or options for alternative medications. Genetic variants include Single Nucleotide Polymorphisms (SNPs). The genetic variants included may be ones that may be tested for with a genetic test.

The client 120 has stored on it a medical client application. The medical client application may comprise a graphical user interface (GUI) and instructions for connecting to the application server 115. A user, such as a doctor, for example, may input a diagnosis into an input field of the medical client application. The medical client application will then cause the communications interface to connect with the application server 115 to request a list of initial therapeutic treatment options for the diagnosis by submitting the diagnosis.

The client 120 may also include an EHR system. The EHR system comprises memory, for storing an EHR, and EHR software for collecting, storing, displaying, and managing the EHR. The EHR may be stored on the client's 120 memory locally in the doctor's office or it may be stored on a server and accessed through a terminal in the doctor's office. The terminal may include a personal computer, a laptop, a tablet computer, or a handheld device such as a smart phone.

The medical client application may be integrated with the EHR software so that the medical client application is included in a display of the EHR. A user may then enter a diagnosis directly in a window displaying the EHR. In other embodiments, however, the medical client application may be stand alone and a user may enter a diagnosis in the medical client application's GUI. The user may be able to log into a website through a secure access portal and access the EHR.

The additional memory 114 also has stored on it a therapeutic options application for execution by the CPU 116 for receiving an indication of a medical condition from the medical client application, and generating an initial therapeutic treatment options list from the database 135. The indication of the medical condition make take various forms, and may include for example a diagnosis, a drug or other medicament, or an explicit indication of the medical condition. The initial therapeutic treatment options list includes selected therapeutic treatment options for treating the medical condition. The therapeutic options application is also configured to obtain one or more patient characteristics from the EHR. The patient characteristics may include a patient's medical history, including one or more of, for example, genetic information, age, gender, functional severity of a disease, pharmacological status with current drugs, medication history, biophysical information, co-morbidities, and allergies. In some embodiments, the therapeutic options application may obtain one or more patient characteristics from multiple sources.

The therapeutic options application is also configured to generate a list of patient specific modifying factors based on the effect of the one or more patient characteristics on the initial therapeutic treatment options from the database 135. The therapeutic options application is also configured to generate a final therapeutic treatment options list by using medication logic trees that include the patient specific modifying factors to modify the initial therapeutic options list and generate therefrom the final therapeutic treatment options list. The final therapeutic options list comprises a list of selected therapeutic treatment options for treating the medical condition provided in the diagnosis. The final therapeutic treatment options list may include one or more suggested changes to medications used to treat other diseases. For example in the presence of the diagnosis of gout the system will check for the presence of drugs likely to make gout worse such as Hydrochlorothiazide. This drug is used to treat hypertension and, if present in any of the final therapeutic treatment options, the system will display an alternative, safer medication using the logic tree for hypertension. The list of final therapeutic treatment options is based on the probability of benefit and harm according to evidence. Rules based on safe prescribing are employed within the process. For example, a rule based on safe prescribing may be that in the situation of having two equally effective medications, where one requires two dosage adjustments based on a biophysical or genetic factor and one does not, the safer option is to use the one without dose adjustments.

The therapeutic options application may be able to link to EHR software based on a variety of platforms. For example, in one embodiment, the therapeutic options application may be able to link to any EHR software that uses HL7 codes. In other embodiments, the therapeutic options application may be able to link to EHR software using other international standards. In yet other embodiments, the therapeutic options application may be able to link to any EHR software. The therapeutic options application does not need to be embedded in an EHR platform in order to connect with it.

The therapeutic options application may send the final therapeutic treatment options list to the medical client application. The final therapeutic treatment options list may be displayed by the medical client application in a display of the EHR. In other embodiments, the final therapeutic treatment options list may be displayed separately from the display of the EHR. For example, the final therapeutic treatment options list may be displayed in a separate window. The list may be, for example, an ordered list from lowest cost to highest cost, or other user determined ordering.

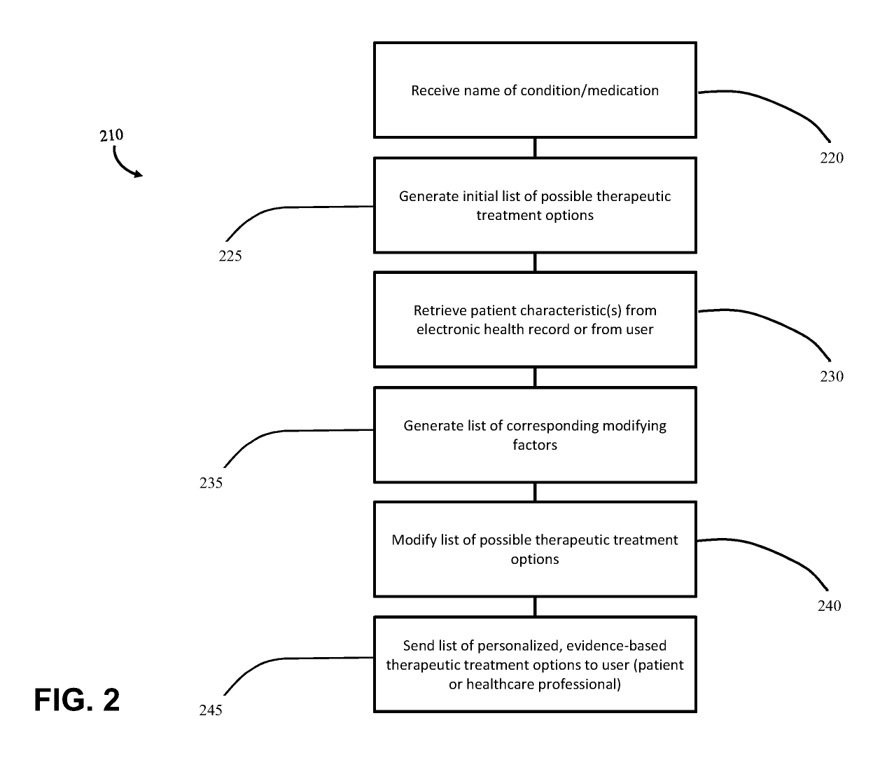

Referring to FIG. 2, a method for providing clinical support 210 is shown. At block 220, an indication of a medical condition, such as a diagnosis or a drug name which is then linked to a diagnosis, is received by the clinical support system 110. The diagnosis may include a name or an indicator of a medical condition. The name may be received from the client 120. For example, a doctor may have input the diagnosis into a GUI of the medical client application running on the medical client 120. The medical client application may be integrated with EHR software being used by the medical client 120. In other applications, the medical client application may be separate from the EHR software. Similarly, in one embodiment, the GUI of the medical client application may be integrated with a display of the EHR. In another embodiment, the medical client application may have a GUI separate from the EHR.

Once the user enters the diagnosis in the medical client application, the CPU of the client 120 provides the coded diagnosis and instructions to send the diagnosis to the application server 115 via the communications interface of the client 120. The communications interface of the client 120 links with the communications interface 126 of the application server 115 and sends the diagnosis to the application server 115. When the client 120 links with the application server 115, the CPU 116 executes the therapeutic options application.

At block 225, an initial list of therapeutic treatment options is generated by the therapeutic options application. The initial therapeutic treatment options list includes selected therapeutic treatment options for treating the medical condition. The initial therapeutic treatment options list is generated from the database 135 stored in the additional memory 114. The database 135 includes a list of medical conditions, therapeutic treatment options for treating each of the medical conditions, and modifying factors for modifying the probabilities of benefit and harm based on a patient's characteristics.

The initial therapeutic treatment options list is generated upon execution of the therapeutic options application by the CPU 116. The CPU 116 parses the database 135 for medical conditions that match the diagnosis. The CPU 116, while executing instructions from the therapeutic options application, will code the diagnosis and match it using a database of diagnostic terminologies to a specific diagnosis. For example “Type 2 Diabetic” will be matched to the disease “Diabetes Mellitus—ICD CM code E11”. The diagnosis code may be dependent on the system being used, ICD10 codes being one of several options. The names of the medical conditions may act as an index for the therapeutic treatment options.

At block 230, the therapeutic options application obtains one or more patient characteristics from the client or directly from the EHR. The CPU 116 instructs the communications interface 126 to connect with the communications interface of the client 120 and send instructions to the medical client application to extract patient characteristics from the EHR and return them to the application server 115. Upon receipt of the instructions by the medical client application, the CPU of the client 120, in accordance with the instructions, retrieves from the EHR all data indexed for the patient being diagnosed. In some embodiments, the CPU may only copy data that is indexed under headings specified by the instructions, such as, for example, genetic information. The client CPU then sends the copied data to the communications interface and instructs the communications interface to send the data to the application server.

In other embodiments, the CPU 116 may send standalone executable instructions directly to an EHR system rather than to the medical client application. The CPU of the client 120 may execute the standalone instructions, copying the requested data from the EHR and sending it back to the application server 115.

The patient characteristics may include genetic information. They may also include at least one of age, gender, functional severity of a disease, pharmacological status with current drugs, medication history, biophysical information, additional medical conditions, and allergies.

At block 235, after receiving the patient characteristics stored in the EHR, the therapeutic options application generates a list of patient specific modifying factors from the list of modifying factors in the database 135 based on the effect of the patient characteristics on the selected therapeutic treatment options.

The list of patient specific modifying factors is generated by the therapeutic options application upon execution of the therapeutic options application by the CPU 116. Upon receiving the list of patient characteristics at the communications interface 126, the CPU 116 searches the database 135 for matching patient characteristics stored in the database 135. For example, in one embodiment, the patient characteristics may represent indexes for associated modifying factors. The CPU 116 copies to the memory 112 the modifying factors that are both associated with the selected therapeutic treatment options and associated with each of the patient characteristics that match the patient characteristics obtained from the EHR, creating the list of patient specific modifying factors. The list of patient specific modifying factors links each selected modifying factor to the therapeutic treatment option it is associated with.

The modifying factors may include factors based on the effect of genetic variants on the therapeutic treatment options. The modifying factors may also include factors based on the effect on the therapeutic treatment options of at least one of; age, gender, functional severity of a disease, pharmacological status with current drugs, medication history, biophysical information, additional medical conditions, and allergies.

At block 240, the therapeutic options application generates a final (modified) therapeutic treatment options list, comprising selected therapeutic treatment options for treating the medical condition. The final therapeutic treatment options list is generated by using the patient specific modifying factors in association with the evidence for effectiveness and harm. To do this, the CPU 116 may go through multiple branches of possible treatments integrating the received patient characteristics with therapeutic evidence to determine the medication options. The resulting therapeutic treatment options are stored in the memory 112 as part of a list.

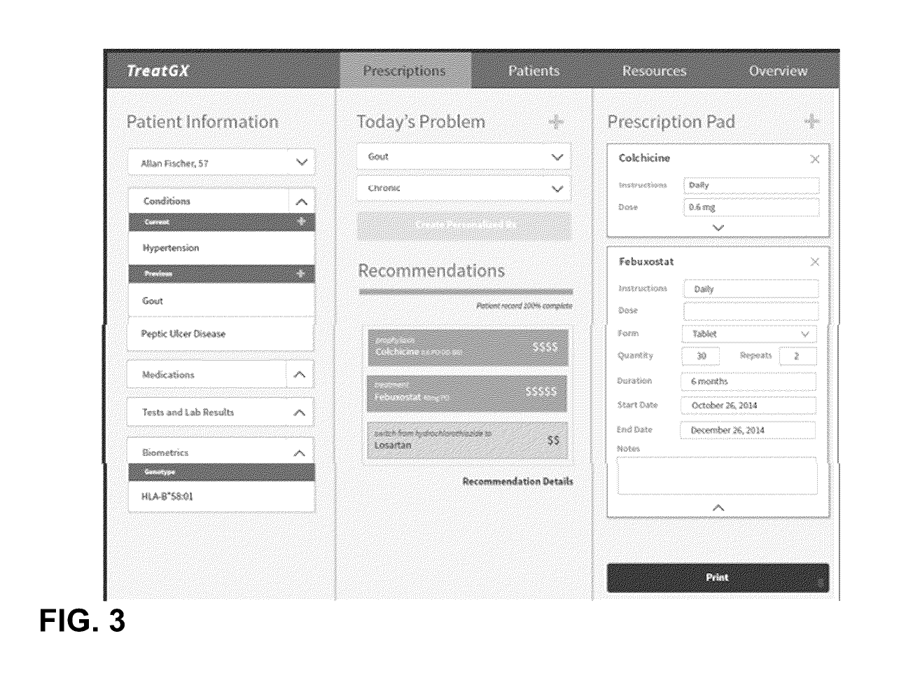

At block 245, the therapeutic options application sends the list of personalized, evidence-based therapeutic treatment options to the client 120. The CPU 116 instructs the communications interface 126 to connect with the communications interface of the client 120 and to send the final therapeutic treatment options list. The final list may be stored with the EHR or separately. The client application may display the final list, either as part of the display of the patient's EHR or separately. Referring to FIG. 3, an example is shown in which the final therapeutic treatment options list is shown as a list of options within a display of a patient's EHR.

Each block of the flowcharts and block diagrams and combinations thereof can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions or acts specified in the blocks of the flowcharts and block diagrams.

In some embodiments, the therapeutic options application may include, for example, a Boolean logic component for modifying the selected therapeutic treatment options based on the total benefits and harms. For example, in certain embodiments, the displayed dosage of a drug may be reduced due to a risk of, for example, renal impairment. However, if a presence of a genetic variant suggests an additional possible harm, in addition to the possibility of renal impairment, then that selected therapeutic treatment option may be removed from the initial therapeutic treatment options list and replaced with other possible therapeutic treatment options that have less possible harms associated with them. In some embodiments, the dosage of the selected therapeutic treatment option may be modified to levels suitable for reducing the risk of harm. Warnings and/or suggestions for a referral for specialist treatment may also be added or substituted for a therapeutic treatment option. In one embodiment, the presence of either an innate harm associated with the selected therapeutic treatment option or patient specific harm associated with the interaction of the selected therapeutic treatment option with one or more patient characteristics causes the therapeutic options application to modify a dosage of the selected therapeutic treatment option using modification guidelines stored in the database 135. However, the presence of an innate harm associated with the selected therapeutic treatment option and at least one patient specific harm may cause the therapeutic options application to remove the selected therapeutic treatment option if there are other suitable therapeutic treatment options available. If there are no other suitable therapeutic treatment options available, then the dosages may be modified. Similarly, if no harms are associated with the selected therapeutic treatment option but there are patient specific harms associated with the interaction of the selected therapeutic treatment option with more than one patient characteristic, so that there are multiple total harms present, the selected therapeutic treatment option may be removed so long as there are other suitable therapeutic treatment options available. In some embodiments, the presence of a single harm may cause the therapeutic options application to remove the associated selected therapeutic treatment option if the harm is on a list of harms in the database 135 that are considered too high. Examples of these are identified within the STOPP criteria.

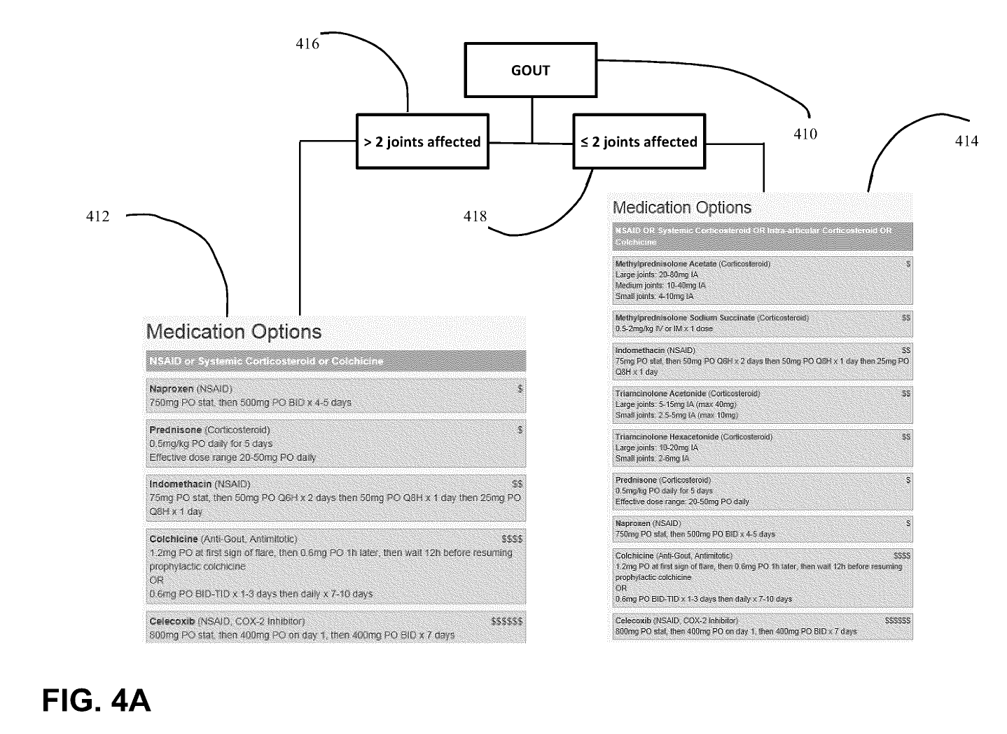

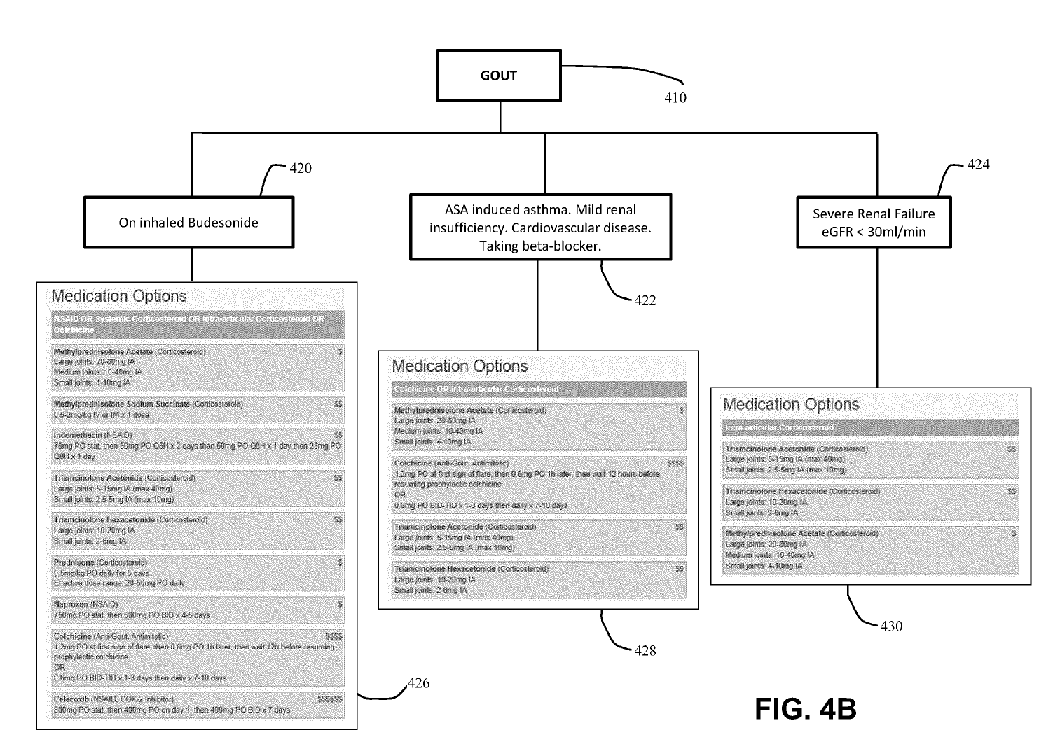

Referring to FIGS. 4A and 4B, an example embodiment for Gout shows the modification of selected therapeutic treatment options for different patient characteristics. For example, in FIG. 4A the system 110 has received a diagnosis input of acute Gout from a user, where symptoms appeared less than 36 hours ago. The therapeutic options application obtains information from the EHR about the functional severity of the patient's Gout. At block 418, the medical client 120 sends information obtained by the medical client application from the EHR that fewer than 3 joints are affected. A list 414 of therapeutic options is provided. If more than two joints are affected, as at block 416, the application generates a list 412 that includes fewer medications as therapeutic treatments options than the list 414. Drugs such as Methylprednisolone Acetate, Triamcinolone Acetate, and Triamcinolone Hexacetonide are not included based on the severity of the medical condition. In this case, the excluded drugs are injectable and injecting drugs into more than two joints may be undesirable. FIGS. 4B and 4B′ again show an example of the clinical support system 110 providing different outcomes for different patient characteristics in a case where a user has provided a diagnosis input of acute Gout. The results for three different situations, which may represent three different patients, is shown. In the first situation, the medical client 120 has sent to the application server 115 medication history 420 from the EHR stored in a memory of the client 120, indicating that the patient is using a Budesonide Inhaler. A list 426 of therapeutic treatment options is provided. Block 422 shows a list of possible patient characteristics that may be obtained from the EHR for another patient that is not using a Budesonide Inhaler. A list of therapeutic options 428 will be generated for a patient that has any of the conditions shown in the list of block 422 and who is not using a Budesonide Inhaler. Block 424 shows the patient characteristics retrieved from the EHR for a patient with severe renal insufficiency. A corresponding list 430 of therapeutic treatment options is generated by the clinical support system based on the patient characteristics shown at block 424. Each of the lists 426, 428, 430 has a different number of therapeutic treatment options for treating an individual with an acute case of Gout. Therapeutic treatment options have been removed or added by the CPU 116 executing the therapeutic options application in each situation based on the additional harms are benefits they offered.

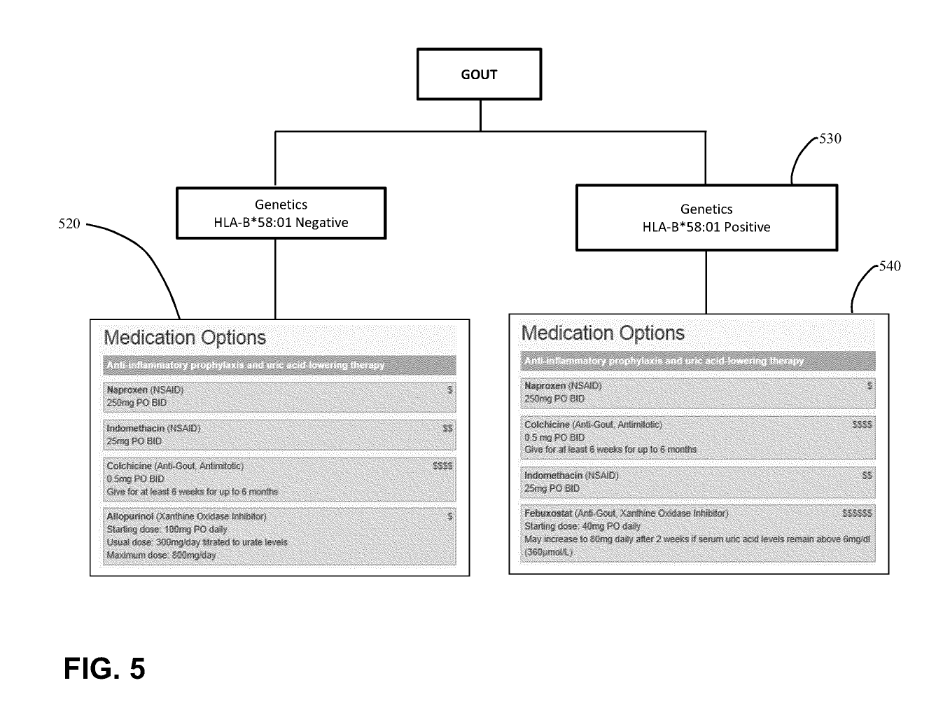

Referring to FIG. 5, an example is shown of the clinical support system 110 providing different outcomes for a diagnosis of chronic Gout for patients with and without the genetic variant HLA-B*58:01. In the first situation, the client 120 has sent to the application server 115 genetic information in a memory of the client 120, indicating that the patient does not have the genetic variant HLA-B*58:01. The application server 115 provides a list 520 of selected therapeutic treatment options. In the second situation, the client 120 has sent to the application server 115 genetic information 530, indicating that the patient has the genetic variant HLA-B*58:01. Based on risk factors (not shown) stored in the database 135 and associated with the genetic variant, the CPU 116 provides a list 540 of selected therapeutic treatment options that excludes the drug Allopurinol, found in the list 520. In another embodiment, the clinical support system 110, upon receiving a diagnosis input, may provide a list of therapeutic treatment options for a variety of patient characteristics. The user may specify the characteristics that they want included. For example, the support system may provide a list that includes therapeutic treatment options for both the presence and absence of a particular genetic variation.

It is contemplated that any part of any aspect or embodiment discussed in this specification can be implemented or combined with any part of any other aspect or embodiment discussed in this specification.

While particular embodiments have been described in the foregoing, it is to be understood that other embodiments are possible and are intended to be included herein. It will be clear to any person skilled pharmaceutical sciences and/or medicine and/or clinical decision support that modifications of and adjustments to the foregoing embodiments, not shown, are possible.

1. A computer-implemented method for providing clinical support, the method comprising:
(a) receiving at a device an indication of a medical condition;
(b) accessing with the device a database comprising a list of medical conditions, a list of therapeutic treatment options for treating each of the medical conditions, a list of patient characteristics, and a list of modifying factors of each patient characteristic on each therapeutic treatment option;
(c) generating at the device an initial therapeutic treatment options list based on the indication of the medical condition, the list of medical conditions and the list of therapeutic treatment options;
(d) receiving at the device at least one patient characteristic;
(e) generating at the device a list of patient specific modifying factors based on the at least one patient characteristic and the list of modifying factors of each patient characteristic on each therapeutic treatment option; and
(f) generating at the device a final therapeutic treatment options list based on the initial therapeutic treatment options list and the list of patient specific modifying factors,
wherein one of the therapeutic treatment options comprises a drug and a dosage regime of the drug, one of the modifying factors comprises an adjustment of the one of the therapeutic treatment options, and the adjustment comprises a modification of the dosage regime.

2. The method of claim 1, wherein each patient characteristic in the list of patient characteristics comprises information on at least one of: a diagnosis, an age, a gender, a functional severity of a disease, a pharmacological status with current drugs, a medication history, biophysical information, an additional medical condition, and an allergy.

3. The method of claim 1, wherein each patient characteristic in the list of patient characteristics comprises genetic information, and wherein the genetic information comprises a genetic variant.

4. The method of claim 1, wherein generating the initial therapeutic treatment options list comprises: identifying in the list of medical conditions a particular medical condition based on the received indication of a medical condition; and using one or more logic trees to identify in the list of therapeutic treatment options one or more particular therapeutic treatment options, the one or more particular therapeutic treatment options being suitable for treating the particular medical condition.

5. The method of claim 1, wherein generating the list of patient specific modifying factors comprises using one or more logic trees to identify in the list of modifying factors one or more particular modifying factors, the one or more particular modifying factors being associated with at least one of the received one or more patient characteristics and a therapeutic treatment option in the initial therapeutic treatment options list.

6. The method of claim, wherein a logic tree identifies associations between one or more of:
(a) a medical condition in the list of medical conditions and a therapeutic treatment option in the list of therapeutic treatment options;
(b) a modifying factor in the list of modifying factors, a patient characteristic in the list of patient characteristics and a therapeutic treatment option in the list of therapeutic treatment options; and
(c) a modifying factor in the list of modifying factors, a patient characteristic in the list of patient characteristics, a therapeutic treatment option in the list of therapeutic treatment options and a medical condition in the list of medical conditions.

7. The method of claim 4, wherein the one or more logic trees are stored in the database.

8.-9. (canceled)

10. The method of claim 1, wherein the adjustment comprises one of the following: a removal of the therapeutic treatment option from the initial therapeutic treatment options list; and a replacement of the therapeutic treatment option in the initial therapeutic treatment options list with another therapeutic treatment option from the list of therapeutic treatment options.

11. The method of claim 1, wherein one of the modifying factors is based on at least one of: evidence of harm linking the patient characteristic to the therapeutic treatment option; and evidence of benefit linking the patient characteristic to the therapeutic treatment option.

12. (canceled)

13. The method of claim 1, wherein receiving the at least one patient characteristic comprises accessing an electronic health record of a patient.

14.-15. (canceled)

16. A system for providing clinical support, the system comprising:
a database comprising a list of medical conditions, a list of therapeutic treatment options for treating each of the medical conditions, a list of patient characteristics, and a list of modifying factors of each patient characteristic on each therapeutic treatment option; and
a device comprising memory and a processor configured to:
receive an indication of a medical condition and at least one patient characteristic;
access the database;
generate:
an initial therapeutic treatment options list based on the indication of the medical condition, the list of medical conditions and the list of therapeutic treatment options;
a list of patient specific modifying factors based on the at least one patient characteristic and the list of modifying factors of each patient characteristic on each therapeutic treatment option; and
a final therapeutic treatment options list based on the initial therapeutic treatment options list and the list of patient specific modifying factors,
wherein one of the therapeutic treatment options comprises a drug and a dosage regime of the drug, one of the modifying factors comprises an adjustment of the one of the therapeutic treatment options, and the adjustment comprises a modification of the dosage regime.

17. The system of claim 16, wherein each patient characteristic in the list of patient characteristics comprises information on at least one of: a diagnosis, an age, a gender, a functional severity of a disease, a pharmacological status with current drugs, a medication history, biophysical information, an additional medical condition, and an allergy.

18. The system of claim 16, wherein each patient characteristic in the list of patient characteristics comprises genetic information, and wherein the genetic information comprises a genetic variant.

19. The system of claim 16, the processor is further configured to: identify in the list of medical conditions a particular medical condition based on the received indication of a medical condition; and use one or more logic trees to identify in the list of therapeutic treatment options one or more particular therapeutic treatment options, the one or more particular therapeutic treatment options being suitable for treating the particular medical condition.

20. The system of claim 16, wherein the processor is further configured to use one or more logic trees to identify in the list of modifying factors one or more particular modifying factors, the one or more particular modifying factors being associated with at least one of the received one or more patient characteristics and a therapeutic treatment option in the initial therapeutic treatment options list.

21. The system of claim 19, wherein a logic tree identifies associations between one or more of:
(a) a medical condition in the list of medical conditions and a therapeutic treatment option in the list of therapeutic treatment options;
(b) a modifying factor in the list of modifying factors, a patient characteristic in the list of patient characteristics and a therapeutic treatment option in the list of therapeutic treatment options; and
(c) a modifying factor in the list of modifying factors, a patient characteristic in the list of patient characteristics, a therapeutic treatment option in the list of therapeutic treatment options and a medical condition in the list of medical conditions.

22. The system of claim 19, wherein the one or more logic trees are stored in the database.

23.-24. (canceled)

25. The system of claim 16, wherein the adjustment comprises one of the following: a removal of the therapeutic treatment option from the initial therapeutic treatment options list; and a replacement of the therapeutic treatment option in the initial therapeutic treatment options list with another therapeutic treatment option from the list of therapeutic treatment options.

26. The system of claim 16, wherein one of the modifying factors is based on at least one of: evidence of harm linking the patient characteristic to the therapeutic treatment option; and evidence of benefit linking the patient characteristic to the therapeutic treatment option.

27. (canceled)

28. The method of claim 16, wherein the processor is further configured to access an electronic health record of a patient.

29.-45. (canceled)
